(19) United States
(12) Patent Application Publication
(10) Pub. No.: US 2017/0143469 A1
(43) Pub. Date: May 25, 2017

(54) REMOVABLE IMPLANT FOR GENERATING A TENDON OR A LIGAMENT

(71) Applicant: ASSISTANCE PUBLIQUE - HOPITAUX DE PARIS, Paris (FR)

(72) Inventor: Marc SOUBEYRAND, Le Kremlin-Bicetre (FR)

(21) Appl. No.: 15/320,854

(22) PCT Filed: Jun. 24, 2015
(86) PCT No.: PCT/EP2015/064246

§ 371 Date: Dec. 21, 2016

(30) Foreign Application Priority Data
Jun. 25, 2014 (FR) 1455926

(51) Int. Cl. A61F 2/08; A61L 27/18; A61L 27/16

(52) U.S. Cl. CPC A61F 2/08; A61L 27/16; A61L 27/18; A61F 2002/0081

(57) ABSTRACT

The present invention relates to a removable implant for generating a tendon or a ligament as a replacement for a torn tendon or ligament, said implant having a hollow tubular body (2) made of a biocompatible and non-resorbable material, with an internal lumen (3) delimited by a lateral wall (4) through which one or more outlet orifices (5) open out to the outer surface of the tubular body via lateral openings, said tubular body opening out, at first and second ends (6-1, 6-2) intended to he fixed to a first and a second member, via first and second end openings (7-1, 7-2), the cumulative surface area of the one or more lateral openings representing more than 10% and less than 25% of the outer surface of said tubular body.

### FIELD OF THE INVENTION

The present invention relates to the field of the production of implants for ligament and tendon reconstruction.

### PRIOR ART

Ligaments are anatomical structures which connect bones to one another. Ligaments provide joint stability while at the same time allowing physiological movements. They are tissue structures attached to the bone at their two ends and composed of fibers (collagen, elastic) oriented in the direction of the mechanical stresses that these tissue structures are subjected to, and of cells (fibroblasts).

Tendons are other anatomical structures which connect a bone to a muscle and which transmit the contraction of the muscle body to the bone. Tendons are essential for active mobilization of the joints. Their histological structure is extremely close to that of ligaments, from which they differ mainly by the proportion of certain molecules such as collagen and elastin.

Ligaments and tendons may be damaged during various pathological situations such as trauma, certain infections, tumors or else inflammatory or degenerative phenomena.

When it is a ligament that is damaged, pulled, by a trauma, this involves the occurrence of a strain. At a more severe stage, the complete loss of the function of the ligaments of a joint results in joint instability potentially responsible for long-term joint pain and destruction (arthrosis). The extreme stage of the loss of ligament function of a joint is dislocation, a situation in which the joint surfaces have definitively and permanently lost their anatomical relationships.

When it is a tendon that is torn, this leads to a loss of motor function of the muscle concerned and of joint mobility, since the contraction of the muscle is not transmitted to the bone.

When a ligament or a tendon is torn, three main therapeutic solutions are carried out.

A minority of patients may tolerate certain ligament ruptures very well. For this small number of patients, it may be that no intervention of surgical nature is carried out. For example, the joint for which the tendon is torn is immobilized until complete spontaneous healing of the ligament or the tendon has taken place. This solution is carried out for example for certain ankle sprains.

According to one alternative, a surgical repair of the tendon or ligament is carried out, when said tendon or ligament cannot heal spontaneously, for example in situations in which the rupture edges are distant from one another. The surgical procedure consists in putting the edges of the tendon or ligament rupture back together end-to-end, and in suturing them. This solution is carried out in particular when a tendon in the hand is cut or when the Achilles tendon is torn.

In other situations, a surgical repair is impossible because the ligament or the tendon heals poorly or because it is too damaged, in particular when the ligament or the tendon is torn. Such a situation is encountered, for example, when the anterior cruciate ligament (ACL) is ruptured.

By way of indication, in the United States of America alone, it is estimated that the annual number of ruptures or of the anterior cruciate ligament (ACL) of the knee is approximately 200 000, including approximately 175 000 patients requiring reconstructive surgery. Furthermore, even though ACL ruptures are the most frequent ligament ruptures, all the other ligaments or tendons of the body may tear and potentially require surgical reconstruction.

Ligament or tendon reconstruction may then be carried out according to a variety of techniques described hereinafter, which have recourse to natural transplants or to synthetic transplants.

Among the techniques using natural transplants, mention is made of a first technique, known as “autograft”. To perform an autograft, also known as autologous graft, an intact ligament or else an intact tendon is used, which is taken from the same patient but from another anatomical site. This technique is also denoted autologous transplantation. According to an allograft technique, a ligament or tendon taken from another individual, generally from a deceased individual, is used. It is a heterologous transplant, which is also known as “allograft”.

With regard to synthetic transplants, they may be categorized in two major categories, respectively (i) totally synthetic transplants and (ii) biosynthetic transplants.

Totally synthetic transplants provide a primary mechanical function of stabilization only. They may thus immediately replace the damaged ligament or tendon (primary resistance). On the other hand, the use of totally synthetic transplants cannot be associated with the initiation of tendon or ligament regrowth. These totally synthetic transplants have the function of cables which allow transmission of the bone-bone (ligament) or bone-muscle (tendon) forces. However, totally synthetic transplants undergo wear which weakens them over time, thereby causing them to rupture. The long-term maintenance of totally synthetic transplants may pose serious health problems for the individuals bearing them, said problems being linked in particular to the release of wear debris. Totally synthetic transplants are described in particular in French patent documents Nos. FR 2 315 825, FR 2 477 009, FR 2 784 020, FR 2 704 421, FR 2 687 911, FR 2 624 724 and FR 2 586 927, in PCT application No. WO 89/10101, in European patent application No. EP 0 233 370 and in American patent application No. US 2011/0190886.

Other synthetic transplants, the biosynthetic transplants, generally consist of a rigid structural body made of synthetic material, which is generally bioresorbable, which contains a support material intended to be colonized by cells in order to regenerate a tendon or a ligament. Biosynthetic transplants provide (i) a primary mechanical function of replacement of the torn ligament or tendon, (ii) a function of artificial extracellular matrix that cells (fibroblasts, stem cells, etc.) will be able to colonize and proliferate therein. After implantation of a biosynthetic transplant in the body of the patient, a neotissue is generated from the cells present in the extracellular matrix support, said neotissue gradually replacing, over time, the mechanical function of the structure made of synthetic material of the transplant. Biosynthetic transplants are described, for example, in French patent documents Nos. FR 2 937 243 and FR 2 937 244, and also in European patent application No. EP 0 454 599.

However, biosynthetic transplants have certain drawbacks, detailed hereinafter. First of all, the production of biosynthetic implants is complex, and consequently lengthy and expensive. This is because the production of an extracellular matrix appropriate for allowing cell colonization and proliferation is a very complex process. Secondly, the proliferation of the neotissue in the synthetic extracellular matrix means that the cells are very intimately attached to the matrix support, thereby ruling out any possibility of removing the biosynthetic transplant once the neotendon or the neoligament is formed. Because the synthetic part of the transplant is kept in place in the patient's body in the long term, there is, as for the totally synthetic transplants, a high risk of release of toxic wear debris harmful to the patient's health. The non-removable nature of the biosynthetic transplant may also be a worry during the occurrence of infections. This is because, in the event of infection, the bacteria preferentially attach to inert tissues, such as the synthetic part of a biosynthetic transplant, which then makes it necessary to surgically remove the transplant in order to cure the infection.

In order to at least partially overcome the above drawbacks of biosynthetic transplants, the prior art has proposed the production of biosynthetic transplants of which the structure degrades over time in the patient's body. However, this new generation of biosynthetic transplants is not itself without economic and technical drawbacks. The design of such implants is complex and potentially very expensive, owing in particular to the quality of the biodegradable materials used. Furthermore, the biodegradable nature of the constituent materials of such biosynthetic transplants means that the mechanical properties of the transplant decrease as it is resorbed, which may lead to an excessively early rupture of the transplant which is exposed to the biomechanical stresses caused by joint movement.

There is a need for medical devices of use in the treatment of a tendon or ligament rupture, which are alternatives to or improvements over the known devices.

### SUMMARY OF THE INVENTION

The present invention relates to a removable implant for generating a tendon or a ligament as a replacement for a torn tendon or ligament, said implant comprising a hollow tubular body made of a biocompatible and non-resorbable material, comprising an internal lumen, delimited by a lateral wall through which one or more outlet orifices open out to the outer surface of the tubular body via lateral openings, said tubular body opening out, at first and second ends intended to be attached to a first and a second member, via first and second end openings, the cumulative surface area of the lateral opening(s) representing more than 10% and less than 25% of the outer surface area of said tubular body.

In certain embodiments, the distance (D) between the two end openings varies from −20% to +50% of the length of said tendon or of said ligament to be replaced.

In certain embodiments, the equivalent surface area of the internal lumen (3) varies from −50% to +50% of the value of the equivalent surface area of said tendon or of said ligament to be replaced.

In certain embodiments, the lateral wall of said tubular body is made of a non-resorbable material chosen from homopolymers or copolymers of silicone, polyurethane, polyethylene, polypropylene, polyamide, polyaryl, fluoropolymers, polyfluoroethylene, polyacrylic acid, polyamide (nylon), polycarbonate, polysulfone, polybutadiene, polybutylene, polyethersulfone, polyetherimide, polypheylene oxide, polymethylpentene, polyvinyl chloride, polyvinylidene chloride, polyphthalamide, polyphenylene sulfide, polyether ether ketone (PEEK), polyimide, poly(methyl methacrylate), or a blend of these polymers.

In certain embodiments, at least one of the ends of the tubular body comprises at least one element for attaching said implant to an organ.

The invention also relates to a process for obtaining an implant as claimed in one of claims 1 to 8 comprising the following steps:

a) measuring a tendon or a ligament to be replaced,

b) producing a tubular body of a removable implant (1), (i) of which the distance (D) between the two end openings varies from −20% to +50% of the length of said tendon or of said ligament determined in step a) and (ii) of which the equivalent surface area of the internal lumen (3) varies from −50% to +50% of the value of the equivalent surface area of said tendon or of said ligament that was determined in step a).

### FIGURES

FIG. 1 illustrates several views of a removable implant according to the invention. FIG. 1A is a diagram of a section of the removable implant along the axis of its length. FIGS. 1B and 1C each represent a view from above the removable implant.

FIG. 2 illustrates the use of a removable implant for generating a ligament. FIG. 2A is a diagram of the knee joint with an intact ligament. FIG. 2B is a diagram of the knee joint with a torn ligament. FIG. 2C is a diagram of the knee joint with a torn ligament on which is represented a surgical opening allowing access to the joint. FIG. 2D is a diagram representing the attachment of one of the removable ends of a removable implant according to the invention at the point of attachment of the future ligament, on an area of bone which has previously undergone freshening. FIG. 2E is a diagram representing the removable implant according to the invention which has been attached at both its ends to the respective points of attachment of the future ligament on each of the bones to be connected.

FIG. 2F is a diagram representing the removable implant according to the invention which is in place, after surgical closure of the skin.

FIG. 3 is an image of a histological section of a newly formed ligament after use and then removal of a removable implant according to the invention.

### DETAILED DESCRIPTION OF THE INVENTION

The present invention provides a removable implant (1) for generating a tendon or a ligament as a replacement for a torn tendon or ligament, said implant comprising a hollow tubular body (2) made of a biocompatible and non-resorbable material, comprising an internal lumen (3), delimited by a lateral wall (4) through which one or more outlet orifices (5) open out to the outer surface of the tubular body (2) via lateral openings, said tubular body (2) opening out, at first and second ends (6-1, 6-2) intended to be attached to a first and a second member, via first and second end openings (7-1, 7-2), the cumulative surface area of the lateral opening(s) (5) representing more than 10% and less than 25% of the outer surface area of said tubular body.

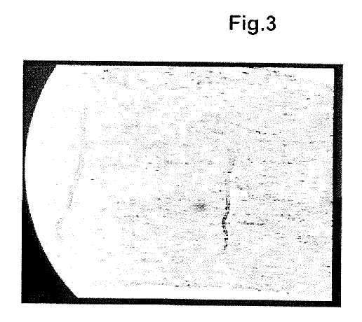

Surprisingly, it is shown according to the invention that a removable implant as specified in the present description allows the generation, ex-nihilo of a new tendon or of a new ligament, as a replacement for a torn tendon or ligament. More specifically, it is shown according to the invention that an implant as defined above is capable of inducing the generation of a new tendon or of a new ligament, the internal surface (8) of the lateral wall (4) of the tubular body (2) guiding the development of a fibrotic tissue along the internal lumen (3), until a neotissue having the shape (macroscopic architecture) and the microscopic architecture of a complete tendon or ligament is formed (see image of FIG. 3).

According to the invention, the shape of the new tendon or of the new ligament is imposed by the shape of the internal lumen (3) of the removable implant (1).

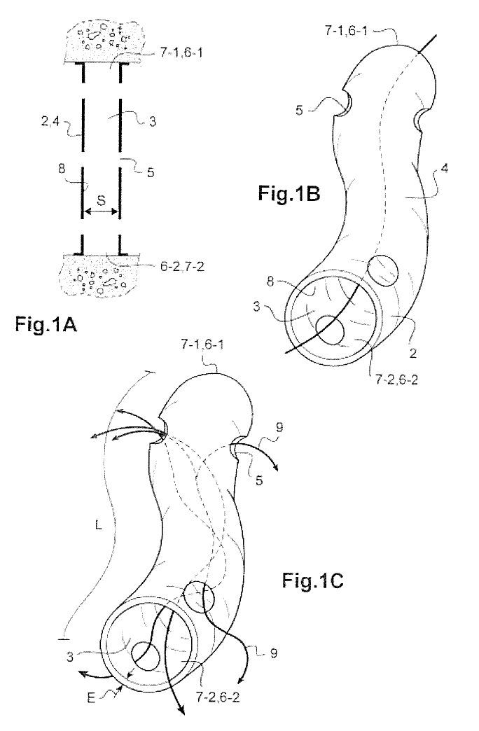

The microscopic architecture of the new tendon or of the new ligament is obtained by virtue of the combination of the technical characteristics of the removable implant. With reference to FIG. 1, for the formation of a new ligament, the removable implant (1) is placed in the patient's body with (i) the first end opening (7-1) placed opposite a first area of bone representing the point of attachment of the future ligament to a first bone and (ii) the second end opening (7-2) placed opposite a second area of bone representing the point of attachment of the future ligament to a second bone, it being understood that each of the first and second areas of bone have been previously freshened, for example by scraping, prior to the placement of the implant. It is specified that each of the two ends (6-1, 6-2) of the implant are attached to each of the two bones. It has been shown according to the invention that the fibrotic tissue generated in the areas of freshening of the bone progresses and is guided along the internal lumen (3) of the implant (I) and that the collagen fibers and the fibroblasts contained in the neotissue are oriented in the direction of formation of the neotissue, as in a normal ligament tissue. The progression of the neotissue in the internal lumen (3) of the tubular body (2) of the implant (1) is made possible because of the lateral openings (5) via which, outside the implant (1), the inflammatory fluids of the tissue flow during healing (represented by the reference (9) in FIG. 1C), thereby enabling the neotissue to progress in the internal lumen (3) without the obstacle that these fluids would provide if they were not discharged to the outside. Moreover, each of the ends (6-1, 6-2) of the implant (1), attached respectively to each of the two bones, are mobile, with respect to one another, and the ligament neotissue is rapidly subjected to the mechanical cycles or stresses of the joint. Thus, the neotissue undergoing construction adapts its development to the physical stimuli to which it is exposed, which brings about a gradual orientation of the microscopic architecture of the neotissue along the axis of the stresses to which it is subjected.

The above description of the formation of a new ligament with the implant (1) also applies to the formation of a new tendon, it being understood that, in this other embodiment, one of the end openings (7-1, 7-2) of the implant (1) is placed opposite an area of muscle or of a residual tendon stump (remainder) representing the point of attachment of the future tendon to a muscle, said area of muscle or residual tendon having previously undergone freshening, for example by scraping the muscle stump.

The presence of the lateral openings (5) is essential for the discharging of the biological fluids (9) contained in the internal lumen (3), thus enabling the progression of the tendon neotissue or of the ligament neotissue in the removable implant (1).

If the cumulative surface area of the lateral opening(s) (5) represents less than 10% of the outer surface area of said tubular body (2), the discharging of the biological fluids (9) is reduced and their presence in the internal lumen (3) prevents the normal progression of the neotissue in said internal lumen (3). In this situation, the duration of complete generation of a new tendon or of a new ligament increases substantially, which is unfavorable to rapid recovery of the animal or of the patient. Furthermore, a reduced speed of discharge of the biological fluids (9) contained in the internal lumen (3) of the removable implant (1) is capable of impairing the microscopic architecture of the neotissue and in particular of affecting the orientation of the collagen fiber bundles, which determine the location of the cell assemblies, in particular the fibroblasts contained in the tissue undergoing formation. Furthermore, if the cumulative surface area of the side opening(s) (5) is less than 10% of the outer surface area of said tubular body (2), the vascularization of the neotissue is reduced, leading to an insufficiency of perfusion of this neotissue and, consequently, reduced development of this neotissue. The general consequence is the formation of a deficient tendon or ligament neotissue, which is not capable of performing the mechanical functions of the tendon or of the ligament that was previously torn.

Conversely, if the cumulative surface area of the lateral opening(s) (5) represents more than 25% of the outer surface area of said tubular body (2), the macroscopic architecture of the new tendon or of the new ligament is capable of being affected because the lateral wall (4) no longer completely performs its role of “mold” for conforming the regenerating tissue which progresses in the internal lumen (3). Furthermore, an excessively large cumulative surface area of the lateral openings (5) compared with the outer surface area of the tubular body (2) is also capable of impairing the mechanical strength properties of the removable implant (1), in particular of the tubular body (2) thereof. In this situation, the mechanical function of the removable implant of temporary replacement of the torn tendon or ligament will not be provided, which would lead to immobilization, or at the very least a considerable impairment, of the animal or of the human patient until complete generation of the new tendon or of the new ligament. In particular, if the cumulative surface area of the lateral opening(s) (5) is greater than 25% of the outer surface area of said tubular body (2), eccentric mechanical stresses, exerted by the tissues surrounding the removable implant, will then be applied to the neoligament or the neotendon undergoing development. These eccentric stresses will interfere with the mechanical stresses parallel to the axis of the tube which ensure the desired longitudinal orientation of the collagen fibers. As it happens, the direction of the mechanical stresses conditions the orientation of the collagen fibers undergoing development in the neoligament/neotendon. Thus, if the cumulative surface area of the lateral opening(s) (5) is greater than 25% of the outer surface area of said tubular body (2), this will thus lead to the production of collagen fibers in eccentric directions which will be detrimental to the overall mechanical properties of the neoligament/neotendon.

In certain embodiments, the lumen of the lateral openings (5), delimited by the thickness of the lateral wall (4), is cylindrical.

In other embodiments, the lumen of the lateral openings (5), delimited by the thickness of the lateral wall (4), is frustoconical, with preferably the largest diameter on the side of the lumen (3), that is to say on the side of the internal surface of the lateral wall (4), and the smallest diameter on the side of the external surface of the lateral wall (4).

Advantageously, the diameter of the lateral openings (5) at the level of the lateral wall (4) varies from 10 μm to 1 mm, better still from 5 μm to 100 μm.

In the embodiments of the removable implant (1) in which the lateral wall (4) has a plurality of outlet orifices (5) through it, said orifices may be located in the lateral wall in a large variety of arrangements.

Whatever the arrangement of the outlet orifices (5) in the lateral wall (4), said orifices (5) are preferentially distributed substantially uniformly over the largest part of the length L of the tubular body (2). A substantially uniform distribution of the outlet orifices (5) is favorable to the development of a tendon or ligament tissue having, over its entire length, an appropriate microscopic architecture.

Preferentially, the axis of each of the outlet orifice(s) (5) is perpendicular to the longitudinal axis of the tubular body (2).

According to one illustrative embodiment, the lateral wall (4) of the tubular body (2) may comprise a plurality of outlet orifices (5) arranged angularly at 120° from one another.

The primary strength of the removable implant (1) enabling the tendon-ligament generation immediately, but temporarily, performs the function of the torn ligament/tendon and is gradually taken over by the neoligament/neotendon undergoing formation. In the end, the removable implant (1) is surgically removed.

As is shown in FIG. 3, the use of a removable implant (1) as specified in the present description made it possible to generate de novo, in the animal, a tissue structure having the macroscopic appearance and the microscopic appearance of a ligament or of a tendon. After removal of the implant (1), a new autologous ligament or a new autologous tendon was generated, which replaces the torn ligament or tendon.

The removable implant (1) has many advantages compared with the known implants, which are listed hereinafter.

Compared with the techniques for reconstruction by autograft, the use of the removable implant according to the invention makes it possible to dispense with the taking of a tendon/ligament from elsewhere in the patient's body. The loss of the physiological function of the tendon/ligament taken, which is intended to replace the torn tendon, is thus avoided. Furthermore, the vascularized nature of the neotendon generated by virtue of the removable implant (1) leads to better healing than with a tendon extracted from its donor site and which is, by definition, devascularized. Likewise, the surgical procedure is simplified since no tendon from the patient is taken for the purpose of an autograft.

Compared with the techniques for reconstruction by allograft, the risk of rejection, and also the risks of transmitting infectious agents originating from the donor tissue, are avoided. Furthermore, as for the autograft techniques, the vascularized nature of the neotendon generated by virtue of the removable implant (1) leads to better healing than with a tendon extracted from its donor site and which is, by definition, devascularized.

Compared with the reconstruction techniques using purely synthetic tendons or ligaments, the live nature (cells) of the neoligament guarantees its survival and the maintenance of its mechanical properties over time. Conversely, a synthetic transplant is inexorably destined to tear, due to wear. Furthermore, the use of the removable implant according to the invention, because it is removed after complete generation of a tendon or of a ligament, does not lead to the formation of wear debris in a patient's body, which debris may seriously harm the patient's health.

Compared with the reconstruction techniques using biotransplants which integrate an extracellular matrix, the implant (1) is surgically removable without damaging the neoligament/transplant, thereby avoiding the complications associated: (i) with the debris from wear of the transplant, (ii) with the surgical treatment in the event of infection: such a complication would impose ablation of the transplant, but if the latter is interlinked with the neotissue (biosynthetic transplant with integrated extracellular matrix), then it is obligatory to remove the neotissue and the transplant, (iii) the simplicity of development: in particular, since the removable implant (1) is completely hollow, the difficulties associated with the design, the manufacture, the use and the cost of an extracellular matrix are not encountered.

With reference to FIG. 1, the length L between the first and second end opening (7-1, 7-2) is determined by the length of the tendon or of the ligament to be replaced. Generally, the average length of a tendon or of a ligament of a man or of an animal is well known to those skilled in the art. Furthermore, the length of the tendon or of the ligament to be replaced in an individual may today be precisely determined, for example by measurement using medical imaging techniques such as magnetic resonance imaging (MRI).

Preferentially, the distance (D) between the two end openings (7-1, 7-2) varies from −20% to +20% of the length of said tendon or of said ligament to be replaced.

By way of illustration, the length L of a removable implant (1) intended to replace the Achilles tendon of an adult human being is approximately 15 centimeters. The length L of a removable implant (1) intended to replace the long flexor tendon of the thumb would be approximately 200 mm.

Likewise by way of illustration, the length L of a removable implant (1) intended to replace a cruciate ligament of the knee of an adult human being is approximately 11 cm.

It is specified that the removable implant (1) may be used for generating new tendons or new ligaments in mammals in general, which encompasses both small rodents and human beings, which means that the length L may be very varied in size.

Thus, the length L between the first and second end openings (7-1, 7-2) of the tubular body (2) may vary from 3 mm to 250 mm, depending on whether the organ to be replaced is a tendon or a ligament and depending on the type of mammal under consideration.

For use of the removable implant (1) for replacing a ligament in an adult human being, the length L may vary from 3 mm to 100 mm.

For use of the removable implant (1) for replacing a tendon in an adult human being, the length L may vary from 50 mm to 250 mm.

As has already been indicated above, the removable implant (1) allows the generation of a tendon or of a ligament of which the tissue structure has the macroscopic appearance, that is to say the same size and the same shape, as the torn tendon or ligament. It is shown according to the invention that the neotissue that is generated and that progresses along the internal lumen (3) is, as it were, “molded” in the latter, the shape of which it takes. Thus, the shape and the size (for example the diameter) of a cross section along the transverse axis of the new tendon or ligament is approximately identical to the shape and the size along the transverse axis of the internal lumen (3) of the removable implant.

Generally, the cross section of the internal lumen of said tubular body, along the transverse axis, has a shape similar to the shape of the transverse cross section of the ligament or tendon to be replaced.

Generally, the cross section (S) (reference S on FIG. 1A) of the internal lumen of said tubular body, along the transverse axis, has an equivalent surface area similar to the equivalent surface area of the transverse cross section of the ligament or tendon to be replaced.

By convention, for the purposes of the present description, the transverse cross section (S) of the internal lumen (3) comprises a plane which is delimited by the internal face of the lateral envelope (4) of the removable implant, said plane having a surface area, also known as “equivalent surface area”, of the cross section (S).

Depending on the type of tendon or ligament that must be replaced, the size of a cross section (S) along the transverse axis of the internal lumen (3) may vary considerably. Likewise, depending on the type of tendon or ligament to be replaced, the shape of a cross section along the transverse axis of the internal lumen (3) may vary, for example depending on whether it is desired to replace a tendon or ligament that is flat or instead of cylindrical shape. Independently of its shape, the size of the cross section along the transverse axis of the internal lumen (3) may thus be expressed by its surface area (or equivalent surface area).

Preferentially, the equivalent surface area of the internal lumen (3) of the implant (1) varies from −20% to +20% of the value of the equivalent surface area of said tendon or of said ligament to be replaced.

By way of illustration, the size of the cross section along the transverse axis of the Achilles tendon of an adult human being is approximately 5 to 6 mm. Since the Achilles tendon is approximately cylindrical, the surface area of its cross section along the transverse axis is approximately 19.63 mm² to 28.26 mm². For use of a removable implant (1) in adult human beings in order to replace a torn Achilles tendon, such an implant of which the cross section of the internal lumen (3) along the transverse axis is approximately cylindrical and has an equivalent surface area ranging from 20 mm² to 30 mm² is preferentially used.

Likewise by way of illustration, the size of the cross section along the transverse axis of a cruciate ligament of the knee of an adult human being is approximately 10 mm. Since the cruciate ligament of the knee is approximately cylindrical, the surface area of its cross section along the transverse axis is approximately 78.5 mm². For use of a removable implant (1) in adult human beings in order to replace a torn cruciate ligament of the knee, such an implant of which the cross section of the internal lumen (3) along the transverse axis is approximately cylindrical and has an equivalent surface area of approximately 80 mm² is preferentially used.

Thus, the equivalent surface area of the internal lumen (3) of the tubular body (2) of the removable implant (1), along the transverse axis, may vary from approximately 0.7 mm² to approximately 1000 mm².

For use of the removable implant (1) for replacing a tendon in an adult human being, the equivalent surface area of the internal lumen (3) of the tubular body (2) of the removable implant (1), along the transverse axis, may vary from approximately −0.7 mm² to approximately 1000 mm².

For use of the removable implant (1) for replacing a ligament in an adult human being, the equivalent surface area of the internal lumen (3) of the tubular body (2) of the removable implant (1), along the transverse axis, may vary from approximately 0.7 mm² to approximately 1000 mm².

As has already been specified, the implant (1) according to the invention is a removable implant, that is to say it is intended to be transiently implanted in the human body, for a period of time sufficient to allow complete generation of a new tendon or of a new ligament, as a replacement for a torn tendon or ligament. Consequently, the removable implant (1) transiently performs the mechanical function of the torn tendon or ligament, which means that the tubular body (2) has a mechanical behavior close to that of the defective tendon or ligament.

After the new tendon or the new ligament has been generated and it replaces the torn tendon or ligament, the removable implant (1) may be removed. The removable implant (1) is thus removed after the new tendon or the new ligament has been completely generated and again performs the mechanical functional of the torn tendon or ligament.

For this reason, the removable implant (1), and in particular the tubular body (2), is made of a biocompatible and non-resorbable material.

For the purposes of the present description, the term “biocompatible material” is intended to mean a material which does not degrade the biological medium in which it is used. In particular, a biocompatible material is not toxic to the organism in which it is implanted, and in particular is free of any cytotoxic property, of any systemic toxicity property or else of any capacity to induce an inflammatory reaction capable of damaging the patient's health.

For the purposes of the present description, the term “non-resorbable material” is intended to mean a material which will not be substantially modified during its residence time in the body of a mammal, in particular in the human body. For the purposes of the invention, the non-resorbable materials encompass materials which are not substantially modified by implantation thereof for a period of six months in the body of a mammal, in particular in the human body. Materials of which the mechanical strength properties are not impaired after a residence time of a period of six months in the body of a mammal, in particular in the human body, are encompassed.

According to one particular aspect of the removable implant (1), the maximum elongation of the tubular body (2) varies from 10% to 50% of its initial length at rest, under the effect of a stretching force of 20 Newtons exerted along its longitudinal axis, which includes from 20% to 50% of its initial length at rest.

In certain embodiments of said removable implant (1), said implant, or at least the lateral wall (4) of the tubular body (2), is made of a non-resorbable material chosen from homopolymers or copolymers of silicone, polyurethane, polyethylene, polypropylene, polyamide, polyaryl, fluoropolymers, polyfluoroethylene, polyacrylic acid, polyamide (nylon), polycarbonate, polysulfone, polybutadiene, polybutylene, polyethersulfone, polyetherimide, polypheylene oxide, polymethylpentene, polyvinyl chloride, polyvinylidene chloride, polyphthalamide, polyphenylene sulfide, polyether ether ketone (PEEK), polyimide, poly(methyl methacrylate), or a blend of these polymers.

With reference to FIG. 1C, the thickness “E” of the lateral wall of the tubular body (2) may be varied, according to the embodiments of the removable implant (1). Generally, the thickness E may depend on the material constituting the lateral wall (4) of the tubular body (2), and in particular on the mechanical properties of said material. Those skilled in the art may easily determine the thickness E, on the basis of their technical general knowledge and the information at their disposal regarding the mechanical properties of a given material, in particular elongation properties of said material under the effect of a tensile strain.

By way of illustration, when the material constituting the lateral wall (4) of the tubular body (2) is a silicone elastomer, such as Silastic®, the thickness E of said lateral wall, for producing an implant intended to replace the Achilles tendon in an adult human being, may vary from 1 mm to 3 mm.

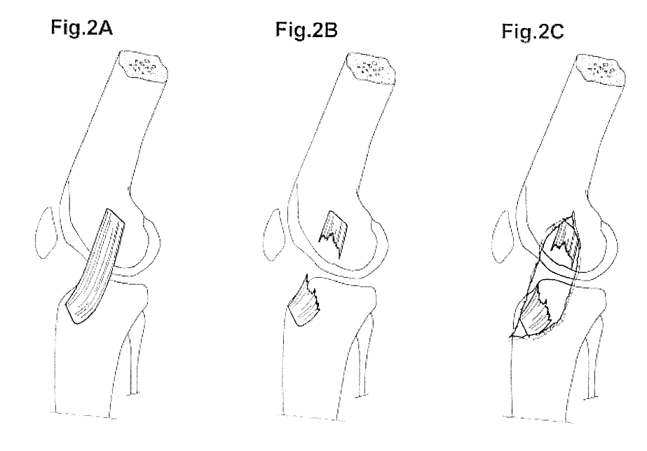

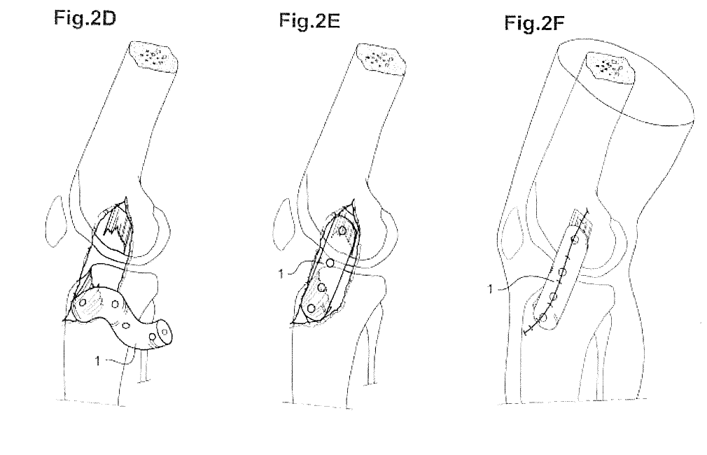

Whatever the embodiment of the removable implant (1), it is essential for said implant to be held in a fixed position in the body of the animal or of the patient, at the site where the end openings (7-1, 7-2) open out on to each of the areas of attachment of the future tendon or ligament, namely (i) the bone areas of attachment if the generation of a new ligament is desired and (ii) the muscle area and the bone area of attachment if the generation of a new tendon is desired (see FIG. 2).

In certain embodiments of the removable implant (1), said implant does not comprise an element for attaching the first and second terminal ends (6-1, 6-2) respectively to a first and a second organ. In these embodiments, a terminal end of the removable implant (1) may be attached to the bone or to the muscle stump by performing a suture. In these embodiments, the surface area of the tubular body (2) which is in immediate proximity to the end openings (6-1, 6-2) preferably does not comprise any lateral opening (5).

In certain other embodiments of the removable implant (1), at least one of the two ends (6-1, 6-2) comprises an element for attaching the implant to an organ, bone or muscle. Said attaching element may be chosen from the numerous attaching elements used in the surgical field and well known to those skilled in the art, such as surgical staples or surgical anchors, or a screwed system.

By way of illustration, a removable implant (1) may comprise, at at least one of its ends (6-1, 6-2), a hollow circular attaching element comprising a lateral wall forming a screw thread on the outside, the inside of said lateral wall delimiting a lumen of the attaching element, and said lumen being in fluidic communication with the internal lumen (3) opening out to the end under consideration (6-1, 6-2) of the tubular body (2). In these embodiments, the removable implant (1) may be attached to the area of bone chosen as attachment point of the future tendon or ligament, on which has previously been hollowed out a cavity of which the lateral wall comprises a screw thread, complementary to the screw thread of the attaching element equipping the removable implant (1).

The removable implant (1) may also comprise, preferably on the internal surface of the lateral wall (4) which is in direct contact with the internal lumen (3), one or more active compounds for improving tissue repair, in particular chosen from antiseptic agents, anti-inflammatory agents, growth factors, polysaccharides such as fucans, proteins such as fibronectin, laminin, or elastin, glycosaminoglycans, proteoglycans, and mixtures thereof. The active compound(s) are present in the form of a layer which coats at least one part of and up to the entire internal surface of the lateral wall (4) of the tubular body (2).

Thus, in certain embodiments of the removable implant (1), the internal surface of the lateral wall (4) comprises a layer of a composition comprising a substance, or a combination of substances, intended to promote the formation of the tendon or ligament tissue. A variety of such substances are known to those skilled in the art, among which are various growth factors. These substances encompass in particular Transforming Growth Factor (TGF), Platelet-Derived Growth Factor (PDGF), Epidermal Growth Factor (EGF), Fibroblast Growth Factor (FGF), Insulin-like Growth Factor (IGF) and Growth and Differentiation Factor (GDF).

With reference to FIG. 2, for replacing a ligament using a removable implant (1) as defined in the present description, a surgical opening is made in the torn ligament (FIG. 2C). Then, after freshening of a first area of bone chosen as first attachment point of the future ligament, one of the ends (6-1) of the removable implant (1) so that the corresponding end opening (7-1) opens out opposite the area of bone representing the first point of attachment, to the bone, of the future ligament (FIG. 2D). Then, after freshening of a second area of bone chosen as second attachment point of the future ligament, the other end (6-2) of the removable implant (1) so that the corresponding end opening (7-2) opens out opposite the area of bone representing the second point of attachment, to the bone, of the future ligament (FIG. 2E). Then, after attachment of the removable implant (1) at the chosen location, the incision is surgically closed (FIG. 2F). The removable implant (1) is kept in the body until complete formation of a new ligament. By way of illustration, the complete formation of a new ligament may be verified by medical imaging techniques, such as MRI. Lastly, after a new ligament has been formed, it is surgically removed according to known methods.

The present invention also relates to a process for obtaining an implant as defined in the present description, said process comprising the following steps:

a) measuring a tendon or a ligament to be replaced,

b) producing a tubular body of a removable implant (1), (i) of which the distance (D) between the two end openings varies from −20% to +50% of the length of said tendon or of said ligament determined in step a) and (ii) of which the equivalent surface area of the internal lumen (3) varies from −50% to +50% of the value of the equivalent surface area of said tendon or of said ligament that was determined in step a).

According to certain embodiments of step a), the measuring of the tendon or of the ligament to be replaced may comprise (i) determining the type of tendon or ligament to be replaced and also the non-human or human mammal involved and the ages thereof, and (ii) determining, by means of the information known in the prior art, the size of the tendon or of the ligament to be replaced.

By way of illustration, for example for measuring a tendon or a ligament of an adult human individual, those skilled in the art may refer to the document

In other embodiments of step a), the measuring of the tendon or of the ligament to be replaced may be carried out by methods using medical imaging techniques, for instance the magnetic resonance imaging (MRI) technique.

The measuring of a tendon or of a ligament to be replaced may be carried out for example by determining the length of said tendon or ligament, this length possibly corresponding to the shortest distance between the two points of attachment of each of its ends to an organ, bone or muscle. The measurement of the tendon or of the ligament advantageously also comprises the measurement of its cross section along its transverse axis. In certain embodiments of step a) of the process, said tendon or said ligament may be likened to a cylinder and the measurement of its cross section along the transverse axis may be the value of its radius or of its diameter.

In other embodiments of step a) of the process, in particular when step a) is carried out using medical imaging techniques such as MRI, the specific geometry of the cross section of the tendon or of the ligament, along its transverse axis, may also be taken into account.

In certain embodiments of the process, the production stage per se of the removable implant (1) may have been carried out beforehand. In these embodiments of the process, a collection of implants is produced beforehand. The tendon or ligament to be replaced is measured, then the implant of which the size, in particular the equivalent surface area of the internal lumen, is closest to the equivalent surface area of the cross section along the transverse axis of the tendon or of the ligament to be replaced is chosen from the collection of implants supplied beforehand.

1. A removable implant for generating a tendon or a ligament as a replacement for a torn tendon or ligament, said implant comprising a hollow tubular body made of a biocompatible and non-resorbable material, comprising an internal lumen delimited by a lateral wall through which one or more outlet orifices open out to the outer surface of the tubular body via lateral openings, said tubular body opening out, at first and second ends intended to be attached to a first and a second member, via first and second end openings, the cumulative surface area of the lateral opening(s) representing more than 10% and less than 25% of the outer surface area of said tubular body.

2. The implant as claimed in claim 1, the distance (D) between the two end openings varying from −20% to +50% of the length of said tendon or of said ligament to be replaced.

3. The implant as claimed in claim 1, the distance D between the first and second end opening of said tubular body ranging from 3 mm to 250 mm.

4. The implant as claimed in claim 1, the equivalent surface area of the internal lumen (3) varying from −50% to +50% of the value of the equivalent surface area of said tendon or of said ligament to be replaced.

5. The implant as claimed in claim 1, the surface area of the lumen of said tubular body, along the transverse axis, ranging from approximately 0.7 mm² to approximately 1000 mm².

6. The implant as claimed in claim 1, the maximum elongation of said tubular body ranging from 10% to 50% of its initial length at rest, under the effect of a stretching force of 20 Newtons exerted along its longitudinal axis.

7. The implant as claimed in claim 1, the lateral wall of said tubular body being made of a non-resorbable material chosen from homopolymers or copolymers of silicone, polyurethane, polyethylene, polypropylene, polyamide, polyaryl, fluoropolymers, polyfluoroethylene, polyacrylic acid, polyamide (nylon), polycarbonate, polysulfone, polybutadiene, polybutylene, polyethersulfone, polyetherimide, polypheylene oxide, polymethylpentene, polyvinyl chloride, polyvinylidene chloride, polyphthalamide, polyphenylene sulfide, polyether ether ketone (PEEK), polyimide, poly(methyl methacrylate), or a blend of these polymers.

8. The implant as claimed in claim 1, at least one of the ends of the tubular body comprising at least one element for attaching said implant to an organ.

9. A process for obtaining an implant as claimed in claim 1, comprising the following steps:
a) measuring a tendon or a ligament to be replaced,
b) producing a tubular body of a removable implant (1), (i) of which the distance (D) between the two end openings varies from −20% to +50% of the length of said tendon or of said ligament determined in step a) and (ii) of which the equivalent surface area of the internal lumen (3) varies from −50% to +50% of the value of the equivalent surface area of said tendon or of said ligament that was determined in step a).
